(19) United States
(12) Patent Application Publication
(10) Pub. No.: US 2012/0245381 A1
(43) Pub. Date: Sep. 27, 2012

(54) METHOD FOR PRODUCING N-ACYLAMINO ACID

(75) Inventor: Toshiaki SUZUKI, Niihama-shi (JP)

(73) Assignee: SUMITOMO CHEMICAL COMPANY, LIMITED, Tokyo (JP)

(21) Appl. No.: 13/424,939

(22) Filed: Mar. 20, 2012

(30) Foreign Application Priority Data
Mar. 23, 2011 (JP) 2011-063710

(51) Int. Cl. C07C 319/20

(52) U.S. Cl. 562/518

(57) ABSTRACT

There is provided a method for producing N-acylamino acid of formula (I):

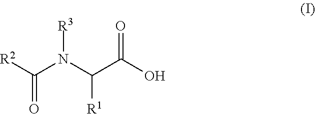

wherein R¹, R² and R³ are the same or different and each independently represents a hydrogen atom, a substituted or unsubstituted hydrocarbyl group, or a substituted or unsubstituted heterocyclic group,
   which comprises supplying an aldehyde compound of formula (II):

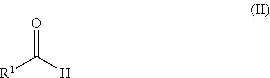

wherein R¹ is as defined above,
   an amide compound of formula (III):

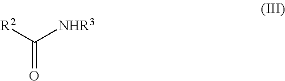

wherein R² and R³ are as defined above,
and a solvent to a reactor in which a solvent, a palladium compound, a halide compound, and carbon monoxide had been charged.

### FIELD OF THE INVENTION

The present invention relates to a method for producing N-acylamino acid, which is useful as raw materials of pharmaceuticals, agrochemicals and methionine.

### BACKGROUND OF THE INVENTION

WO98/04518 teaches a carbonylation reaction of an aldehyde compound, an amide compound in a solvent in the presence of a mixture, as catalyst, of palladium compound, halide ion and an acid charged in a reactor to which carbon monoxide is introduced to produce N-acylamino acid. The reaction method is not always satisfactory in that it does not proceed readily as disclosed.

### SUMMARY OF THE INVENTION

An objective of the invention is to provide a method for producing a N-acylamino acid readily in a good reproducible manner.

The present invention provides:

1. a method for producing N-acylamino acid of formula (I):

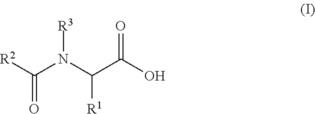

wherein R¹, R² and R³ are the same or different and each independently represents a hydrogen atom, a substituted or unsubstituted hydrocarbyl group, or a substituted or unsubstituted heterocyclic group,
which comprises supplying an aldehyde compound of formula (ID:

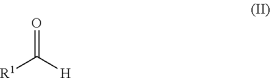

wherein R¹ is as defined above,
an amide compound of formula (III):

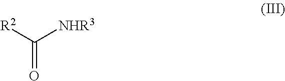

wherein R² and R³ are as defined above,
and a solvent to a reactor in which a solvent, a palladium compound, a halide compound, and carbon monoxide had been charged;
2. a method according to item 1, wherein the amount of the solvent that had been charged in the reactor is 50 to 90% by mass of the total amount of the solvent to be supplied and the charged solvent;
3. a method according to item 1 or 2, wherein the halide compound is a halide compound selected from the group consisting of an alkali metal halide, ammonium halide, and a quaternary ammonium halide;
4. a method according to item 1 or 2, wherein the halide compound is an alkali metal halide;
5. a method according to any one of items 1 to 4, wherein the palladium compound is palladium halide;
6. a method according to any one of items 1 to 5, wherein the solvent is an aprotic polar solvent;
7. a method according to any one of items 1 to 5, wherein the solvent is 1-methyl-2-pyrrolidinone;
8. a method for producing N-acylamino acid of formula (I) as defined above, which comprises bringing a catalytic amount of palladium compound and a halide compound into contact in a solvent under an atmosphere of carbon monoxide to produce a catalyst mixture, and supplying the aldehyde compound of formula (II) as defined above, and the amide compound of formula (III) as defined above to the resulting mixture;
9. a method according to item 8, wherein R¹ is a hydrogen atom, an alkyl group, an alkylthioalkyl group, an alkenyl group, an aryl group, or an aralkyl group, R² and R³ are the same or different and independently represent a hydrogen atom, an alkyl group, an aryl group, or an aralkyl group;
10. a method according to any one of items 1 to 8, wherein R¹ is a hydrogen atom, a (C1-C6)alkyl group, a (C1-C4)alkyl-thio-(C1-C4)alkyl group, a (C2-C4)alkenyl group, a (C6-C14)aryl group, or a (C7-C₁₄)aralkyl group, and R² and R³ are the same or different and each independently represent a hydrogen atom, a (C1-C6)alkyl group, a (C6-C14)aryl group, or a (C7-C14)aralkyl group; and
11. a method according to any one of items 1 to 8, wherein R¹ is a 2-methylthioethyl group, R² is a methyl group, and R³ is a hydrogen atom.

### DESCRIPTION OF THE PREFERRED EMBODIMENTS

A description will be made to the substituent represented by R¹, R² and R³ groups.

Examples of the unsubstituted hydrocarbyl group include, for example, an alkyl grouop, alkenyl group, a cycloalkyl group, a cycloalkenyl group, an alkynyl group, and an aryl group.

Examples of the alkyl group include, for example, (C1-C24)alkyl groups such as methyl, ethyl, propyl, isopropyl, butyl, isobutyl, sec-butyl, t-butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, icosyl, henicosyl, docosyl, tricosyl, and tetracosyl groups.

Examples of the alkenyl group include, for example, (C2-C24)alkenyl groups such as vinyl, allyl, 2-methylallyl, isopropenyl, 1-propenyl, 1-butenyl, 2-butenyl, 3-butenyl, 1-methyl-1-propenyl, 1-methyl-2-propenyl, 2-methyl-1-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-hexenyl, 2-hexenyl, 3-hexenyl, 4-hexenyl, 5-hexenyl, 1-methyl-1-pentenyl, 2-methyl-1-pentenyl, 4-methyl-3-pentenyl, 2-ethyl-1-butenyl, 2-heptenyl, 2-octenyl, 2-nonenyl, 2-decenyl, 2-undecenyl, 2-dodecenyl, 2-tridecenyl, 2-tetradecenyl, 2-pentadecenyl, 2-hexadecenyl, 2-heptadecenyl, 2-oxtadecenyl, 2-nonadecenyl, 2-icosenyl, 2-henicosenyl, 2-docosenyl, 2-tricosenyl, and 2-tetracesenyl groups.

Examples of the alkynyl group include, for example, (C2-C24)alkynyl groups such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-3-butynyl, 2-methyl-3-butynyl, 1-hexynyl, 2-heptynyl, 2-octynyl, 2-nonynyl, 2-decynyl, 2-undecynyl, 2-dodecynyl, 2-tridecynyl, 2-tetradecynyl, 2-pentadecynyl, 2-haexadecynyl, 2-heptadecynyl, 2-octadecynyl, 2-nonadecynyl, 2-icosynyl, 2-henicosynyl, 2-docosynyl, 2-tricosynyl, and 2-tetracosynyl, groups.

Examples of the cycloalkyl group include, for example, (C3-C8)cycloalkyl groups such as cyclopropyl, cyclobutyl, cyclopentyl, cyclohexyl, cycloheptyl, and cyclooctyl groups.

Examples of the cycloalkenyl groups include, for example, (C3-C8)cycloalkenyl groups such as cyclopropenyl, cyclobutenyl, cyclopentenyl, cyclohexenyl, cycloheptenyl, and cyclooctenyl groups.

Examples of the aryl group include, for example, (C6-C18)aryl groups such as phenyl, naphthyl, anthranyl, phenanthroryl, tolyl, and xylyl groups.

Examples of the heterocyclic group include, for example, (C3-C9)heteroaryl group such as pyridyl, quinolyl, pyrrolyl, imidazolyl, furyl, indolyl, thienyl, and oxazolyl groups.

The alkyl group, the alkenyl group and the alkynyl group represented by R¹, R², or R³ may have substituent(s). Examples of the substituents include, for example, halogen atom(s), (C3-C6)cycloalkyl group, (C1-C4)alkoxy group, (C1-C4)alkyl-thio group, (C3-C4)alkenyloxy group, (C7-C20)aralkyloxy group, (C6-C14)aryl group, (C6-C18)aryloxy group, (C2-C7)alkanoyl group, (C7-C19)aryl-carbonyl group, (C2-C7)alkanoyloxy group, (C7-C19)aryl-carbonyloxy group, (C2-C7)alkanoylamino group, (C1-C6)alkyl-sulfonylamino group, (C2-C6)alkoxy-carbonylamino group, benzylcarbonylamino group, (C6-C18)aryl-sulfonylamino group, and aminocarbonyl group, (C1-C6)alkoxy-carbonyl group.

Examples of the alkyl group substituted with (C6-C14)aryl group include, for example, (C7-C20)aralkyl group (e.g., benzyl, phenethyl, 3-phenylpropyl, benzhydryl, trityl, triphenylethyl, (1-naphthyl)methyl, and (2-naphthyl)methyl groups).

The cycloalkyl group, the cycloalkenyl group, and the aryl group represented by R¹, R², or R³ may have substituent(s). Examples of the substituents include, for example, halogen atom(s), C3-C6cycloalkyl group, (C1-C4)alkoxy group, (C1-C4)alkyl-thio group, (C3-C4)alkenyloxy group, (C7-C20)aralkyloxy group, (C6-C18)aryl group, (C6-C18)aryloxy group, (C2-C7)alkanoyl group, (C7-C19)aryl-carbonyl group, (C2-C7)alkanoylamino group, (C1-C6)alkyl-sulfonylamino group, (C2-C6)alkoxy-carbonylamino group, benzylcarbonylamino group, (C6-C18)aryl-sulfonylamino group, aminocarbonyl group, (C1-C6)alkoxy-carbonyl group, (C2-C6)alkenyl group as defined above, and (C7-C20)aralkyl group.

The aryl group represented by R¹, R², or R³ may be substituted by hydroxyl group(s) or protected hydroxyl group(s).

The heterocyclic group represented by R¹, R², or R³ may have a substituent(s). Examples of the substituent(s) include, for example, halogen atom(s), (C1-C6)alkyl group, (C3-C6)cycloalkyl group, (C1-C4)alkoxy group, (C1-C4)alkyl-thio group, (C3-C4)alkenyloxy group, (C7-C20)aralkyloxy group, (C6-C18)aryl group, (C6-C18)aryloxy group, (C2-C alkanoyl group, (C7-C19)aryl-carbonyl group, (C2-C7)alkanoylamino group, (C1-C6)alkyl-sulfonylamino group, (C2-C6)alkoxy-carbonylamino group, benzylcarbonylamino group, (C6-C18)aryl-sulfonylamino group, aminocarbonyl group, (C2-C6)alkenyl group, and (C7-C20)aralkyl group.

Specific examples of the aldehyde compound of formula (II) include, for example, formaldehyde, acetaldehyde, propionaldehyde, butyraldehyde, valeraldehyde, 3-(methylthio)propionaldehyde, 2-ethylhexanal, isobutyraldehyde, furfural, crotonaldehyde, acrolein, benzaldehyde which may be substituted with the substituent as described above for the substituents that may be present on the aryl group above, phenylacetoaldehyde, 2,4-dihydroxyphenylacetaldehyde, gyoxalic acid, and α-acetoxypropionaldehyde. Preferred aldehyde compound of formula (II) is 3-(methylthio)propionaldehyde.

Specific examples of the amide compound of formula (III) include, for example, acetamide, benzamide, propionamide, N-methylacetamide, aliphatic amide, acrylamide, cinnamylamide, phenylacetamide, and acetanilide. Preferred is acetamide.

The amide compound of formula (III) is preferably used in the amount of 1 mol or more, more preferably 1.05 to 2 moles per mol of the aldehyde compound of formula (I).

Followings are preferable examples of the combinations of the aldehyde compound of formula (II) and the amide compound of formula (III).

A combination of the aldehyde compound of formula (II), wherein R¹ represents a hydrogen atom, an alkyl group, an alkylthioalkyl group, an alkenyl group, an aryl group, or an aralkyl group, and the amide compound of formula (III), wherein R² and R³ are the same or different and each independently represent a hydrogen atom, an alkyl group, an aryl group, or an aralkyl group.

More preferable combinations include the aldehyde compound of formula (II) wherein R¹ represents a hydrogen atom, an (C1-C6)alkyl group, an (C1-C4)alkyl-thio-(C1-C4)alkyl group, an (C2-C4)alkenyl group, an (C6-C12)aryl group, or an (C7-C14)aralkyl group, and amide of formula (III), wherein R² and R³ are the same or different and each independently represent a hydrogen atom, an (C1-C6)alkyl group, an (C6-C12)aryl group, or an (C7-C14)aralkyl group.

Yet more preferable combination is the aldehyde compound of formula (II), wherein R¹ represents 2-methylthioethyl group, and the amide of formula (III), wherein R² represents a methyl group, and R³ represents a hydrogen atom.

Examples of the solvent that may be used include, for example, protic polar solvent, aprotic polar solvent, and ionic liquid. Preferred is a aprotic polar solvent. Examples of the protic polar solvent include, for example, acetic acid, methanol, ethanol, isopropanol.

Examples of the aprotic polar solvent include, for example, dioxane, tetrahydrofuran, N-methylpyrrolidinone, N-ethylpyrrolidinone, 1,3-dimethyl-2-imidazolidinone, dimethyl sulfoxide, ethyleneglycol dimethyl ether, acetone, ethyl acetate, acetonitorile, benzonitrile, t-butyl methyl ether, dibutyl ether, sulfolane, N,N-dimethylformamide, N,N-dimethylacetamide, toluene. Preferred is N-methylpyrrolidinone. The solvent may be used alone or two or more of them may be used together.

The solvent is preferably used in the amount of 0.5 to 20 parts by mass, more preferably 2 to 10 parts by mass per part by mass of the aldehyde compound of formula (II). When two or more solvents are combined, the total amounts of the solvents are preferably set within the prescribed amount range.

Palladium compounds are used as a catalyst component. Examples of the palladium compound include, for example, divalent palladium compounds such as palladium chloride(II), palladium bromide(II), palladium iodide (II), palladium nitrate (II), palladium sulfate (II), palladium acetate (II); zero valent palladium compounds such as tris(dibenzylideneacetone)dipalladium(0), bis(dibenzylideneacetone)palladium(0), dipalladium(0)tris(dibenzylideneacetone-chloroform); and palladium phosphine complex such as a complex comprising the divalent palladium compound as described above and a phosphine compound (e.g., triphenylphosphine, tritolylphosphine, bis-(diphenylphosphino)-ethane). The palladium compound may be used in a form of a formed catalyst (formed palladium compound catalyst), or in a form of a carrier-supported catalyst (palladium compound-supported catalyst), or may be supported on a polymer.

The palladium compound is preferably used in the amount of 0.0001 to 0.5 mol, more preferably 0.001 to 0.05 mol per mol of the aldehyde compound of formula (II).

In the carbonylation reaction of the aldehyde compound of formula (II) and the amide compound of formula (III) with carbon monooxide, the halide compound is preferably used together with the palladium compound. Preferably employed halide compound is a halide compound selected from the group consisting of alkali metal halide, ammonium halide, and quaternary ammonium halide.

Preferred is an alkali metal halide selected from the group consisting of lithium iodide, sodium iodide, potassium iodide, lithium bromide, sodium bromide, potassium bromide, lithium chloride, and potassium chloride. More preferred is lithium bromide.

A catalytic amount of the halide compound is usually employed. The halide compound is preferably used in the amount of 0.01 to 0.5 mol, more preferably 0.2 to 0.4 mol per mol of the aldehyde compound of formula (II).

The carbonylation reaction of the aldehyde compound of formula (II), and the amide compound of formula (III) under carbon monoxide atmosphere proceed, without requiring an acid such as inorganic acid (e.g., hydrogen halide, sulfuric acid, phosphoric acid), or an organic acid. In some embodiments of the invention, the reaction can be carried out in the absence of an acid.

The method of the invention, the carbonylation reaction, is typically carried out as follows.

The production process comprises supplying an aldehyde compound of formula II as defined above, an amide compound of formula (III) as defined above, and a solvent to a reactor in which a solvent, a palladium compound, a halide compound, and carbon monoxide had been charged, thereby N-acylamino acid of formula I is produced.

The solvent, palladium compound, halide compound and carbon monoxide are charged in a reactor where the charging order thereof is not limited. Preferably, after the solvent, palladium compound, halide compound had been charged in a reactor, carbon monoxide is charged thereto. Then, aldehyde compound of formula (II), the amide compound of formula (III) as reactants, and the solvent are supplied (fed) each independently or together with other reactant(s) or solvent, in a form of co-feed, or supplied all together as a single solution containing the reactants and the solvent to the reactor where the solvent, palladium compound, halide compound and carbon monoxide had been charged beforehand. Preferably, the aldehyde compound of formula (II), the amide compound of formula (III), and the solvent are supplied to a reactor where the solvent, palladium compound, halide compound and carbon monoxide had been charged beforehand. Preferably, the amount of the solvent that had been charged in the reactor is 50 to 90% by mass of the total amount of the charged solvent and the solvent to be supplied.

The supplying of the aldehyde compound of formula (II), and the amide compound of formula (III) each may be continuous with or without any intervals or intermittence. The starting of supplying of each of the aldehyde compound of formula (II) and the amide compound of formula (III), and termination of supplying of each of the aldehyde compound of formula (II) and the amide compound of formula (III) do not have to coincide exactly, but may be varied as long as the adverse affect does not arise.

The aldehyde compound of formula (II) is supplied, preferably as being cooled, whereby reaction between two aldehyde molecules, aldol condensation, can be suppressed, which means that byproduct(s) derived from the condensate can be controlled. The cooling temperature of the aldehyde compound of formula (II) may be suitably set for the aldehyde, and is preferably −20 to 5° C.

The reaction temperature for producing N-acylamino acid of formula (I) is preferably at 60 to 140° C., more preferably 80 to 120° C. The reaction may be carried out under normal pressure, preferably under pressure of 0.1 to 25 MPa (absolute), more preferably under pressurized pressure of 5 to 15 MPa (absolute). The reaction can be carried out in a batch-wise manner, semi-batch-wise manner, or continuous manner.

After-treatment of the reaction mixture containing N-acylamino acid of formula (I) thus produced may be suitably selected, and the product can be purified by an optional treatment such as washing, distillation, or crystallization, if necessary, for various use thereof.

The production method can be also carried out by the steps of bringing a catalytic amount of the palladium compound and the halide compound into contact in a solvent under an atmosphere of carbon monoxide, preferably pressurized carbon monoxide, to produce a catalyst mixture, and supplying the aldehyde compound of formula (II) as defined above, and the amide compound of formula (III) as defined above to the resulting mixture. Such embodiments of the production method embrace possible combinations of the reaction conditions and the manners of the reactions as described above.

Next, the invention is explained by way of examples but is not to be construed to be limited by the examples.

The content of N-acetylmithione, which corresponds to the compound of formula (I), wherein R¹ is methylthioethyl, R² is hydrogen atom, and R³ is a methyl group, was analyzed by high-performance liquid chromatography using Internal Standard method, and the yield was calculated from analyzed values.

### High-Performance Liquid Chromatography Analysis

HPLC Apparatus: Agilent LC-1100, produced by Agilent Technologies.
Column: Scherzo C18(4.6 mm Φ×150 mm, particle diameter: 3 μm; product of Imtakt)
Eluating Sol. Solution A: 0.1% trifluoroacetic acid in water
   Solution B: 0.1% trifluoroacetic acid in acetonitrile

Amount ratio (by vol) of Solution B to the total amount of Solutions A and B, as defined by B % was employed as follows:

B % (min): 5%/0 min−5%/5 min−90%/25 min−90%/30 min−5%/30.1 min−5%/40 min.
Flow rate: 1.0 ml/min
Oven Temperature: 40° C.
UV Detector: 210 nm
Rinse solution: H₂O/acetonitrile=1/1(vol/vol)

### Example 1

A reactor made of stainless steel equipped with a thermocouple, stirrer, gas-supplying line, and liquid-supplying line was charged with 0.33 g (0.0013 mol) of palladium bromide(II), 1.54 g (0.0175 mol) of lithium bromide and 41.20 g of 1-methyl-2-pyrrolidinone (80% by mass of the total amount of 1-methyl-2-pyrrolidinone), and the resulting mixture was stirred and the gas-phase of the reactor was pressurized with carbon monoxide gas by 10 MPa (gauge pressure). Then, the temperature of the reaction mixture was raised to 98 to 102° C. under stirring where the pressure inside the reactor was found to be 10 MPa (gauge pressure). Next, to the reactor was drop-wise added over 3 hrs a solution of mixture of 5.26 g (0.050 mol) of 3-(methylthio)propionaldehyde, which corresponds to the aldehyde compound of formula (II) wherein R¹ is methlythio group, and 3.01 g (0.050 mol) of acetamide, which corresponds to the amide compound of formula (III), wherein R² is a methyl group and R³ is hydrogen atom, in 10.30 g of 1-methyl-2-pyrrolidinone, which corresponds to 20% by mass of the total amount of 1-methyl-2-pyrrolidinone used in this reaction. After the addition, the reaction mixture was kept under stirring for 3 hrs at 98 to 102° C., and then cooled to 5 to 35° C. to give 60.92 g of a solution of N-acetylmethionine in 1-methyl-2-pyrrolidinone. The high-performance liquid chromatography analysis of the solution showed that the yield of N-acetylmethionine based on 3-(methylthio)propionaldehyde was 82.74%.

### Example 2

A reactor made of stainless steel equipped with a thermocouple, stirrer, gas-supplying line, and liquid-supplying line was charged with 0.33 g (0.0013 mol) of palladium bromide(II), 1.54 g (0.0175 mol) of lithium bromide and 41.20 g of 1-methyl-2-pyrrolidinone (80% by mass of the total amount of 1-methyl-2-pyrrolidinone), and the resulting mixture was stirred and the gas-phase of the reactor was pressurized with carbon monoxide gas by 10 MPa (gauge pressure). Then, the temperature of the reaction mixture was raised to 98 to 102° C. under stirring where the pressure inside the reactor was found to be 10 MPa (gauge pressure). Next, to the reactor was drop-wise added over 2 hrs a solution of mixture of 5.26 g (0.050 mol) of 3-(methylthio)propionaldehyde, and 3.01 g (0.050 mol) of acetamide in 10.30 g of 1-methyl-2-pyrrolidinone, which corresponds to 20% by mass of the total amount of 1-methyl-2-pyrrolidinone used in this reaction. After the addition, the reaction mixture was kept under stirring for 4 hrs at 98 to 102° C., and then cooled to 5 to 35° C. to give 60.34 g of a solution of N-acetylmethionine in 1-methyl-2-pyrrolidinone. The high-performance liquid chromatography analysis of the solution showed that the yield of N-acetylmethionine based on 3-(methylthio)propionaldehyde was 77.84%.

### Example 3

A reactor made of stainless steel equipped with a thermocouple, stirrer, gas-supplying line, and liquid-supplying line was charged with 0.67 g (0.0025 mol) of palladium bromide(II), 3.07 g (0.035 mol) of lithium bromide and 41.20 g of 1-methyl-2-pyrrolidinone (80% by mass of the total amount of 1-methyl-2-pyrrolidinone), and the resulting mixture was stirred and the gas-phase of the reactor was pressurized with carbon monoxide gas by 10 MPa (gauge pressure). Then, the temperature of the reaction mixture was raised to 98 to 102° C. under stirring where the pressure inside the reactor was found to be 10 MPa (gauge pressure). Next, to the reactor was drop-wise added over 3 hrs a solution of mixture of 10.52 g (0.10 mol) of 3-(methylthio)propionaldehyde, and 6.03 g (0.10 mol) of acetamide in 20.60 g of 1-methyl-2-pyrrolidinone, which corresponds to 20% by mass of the total amount of 1-methyl-2-pyrrolidinone used in this reaction. After the addition, the reaction mixture was kept under stirring for 3 hrs at 98 to 102° C., and then cooled to 5 to 35° C. to give 81.27 g of a solution of N-acetylmethionine in 1-methyl-2-pyrrolidinone. The high-performance liquid chromatography analysis of the solution showed that the yield of N-acetylmethionine based on 3-(methylthio)propionaldehyde was 77.80%.

### Comparative Example 1

A reactor made of stainless steel equipped with a thermocouple, stirrer, gas-supplying line, and liquid-supplying line was charged with 5.26 g (0.050 mol) of 3-(methylthio)propionaldehyde, 3.01 g (0.050 mol) of acetamide, 0.33 g (0.0013 mol) of palladium bromide(II), 1.54 g (0.0175 mol) of lithium bromide and 51.50 g of 1-methyl-2-pyrrolidinone (100% by mass of the total amount of 1-methyl-2-pyrrolidinone), and the resulting mixture was stirred and the gas-phase of the reactor was pressurized with carbon monoxide gas by 10 MPa (gauge pressure). Then, the temperature of the reaction mixture was raised to 98 to 102° C. under stirring where the pressure inside the reactor was found to be 10 MPa (gauge pressure). Next, the reaction mixture was kept under stirring for 4 hrs at 98 to 102° C., and then cooled to 5 to 35° C. to give 61.07 g of a solution of N-acetylmethionine in 1-methyl-2-pyrrolidinone. The high-performance liquid chromatography analysis of the solution showed that the yield of N-acetylmethionine based on 3-(methylthio)propionaldehyde was 52.22%.

### Comparative Example 2

A reactor made of stainless steel equipped with a thermocouple, stirrer, gas-supplying line, and liquid-supplying line was charged with 5.26 g (0.050 mol) of 3-(methylthio)propionaldehyde, 3.01 g (0.050 mol) of acetamide, 1.04 g (0.0013 mol) of a complex of palladium bromide(II) and triphenylphosphine, 1.54 g (0.0175 mol) of lithium bromide and 51.50 g of 1-methyl-2-pyrrollidinone (100% by mass of the total amount of 1-methyl-2-pyrrolidinone), and the resulting mixture was stirred and the gas-phase of the reactor was pressurized with carbon monoxide gas by 10 MPa (gauge pressure). Then, the temperature of the reaction mixture was raised to 98 to 102° C. under stirring where the pressure inside the reactor was found to be 10 MPa (gauge pressure). Next, the reaction mixture was kept under stirring for 4 hrs at 98 to 102° C., and then cooled to 5 to 35° C. to give 61.16 g of a solution of N-acetylmethionine in 1-methyl-2-pyrrolidinone. The high-performance liquid chromatography analysis of the solution showed that the yield of N-acetylmethionine based on 3-(methylthio)propionaldehyde was 46.98%.

### Comparative Example 3

A reactor made of stainless steel equipped with a thermocouple, stirrer, gas-supplying line, and liquid-supplying line was charged with 5.26 g (0.050 mol) of 3-(methylthio)propionaldehyde, 3.01 g (0.050 mol) of acetamide, 0.33 g (0.0013 mol) of palladium bromide(II), 1.54 g (0.0175 mol) of lithium bromide, 0.05 g (0.00051 mol) of sulfuric acid and 51.50 g of 1-methyl-2-pyrrolidinone (100% by mass of the total amount of 1-methyl-2-pyrrolidinone), and the resulting mixture was stirred and the gas-phase of the reactor was pressurized with carbon monoxide gas by 10 MPa (gauge pressure). Then, the temperature of the reaction mixture was raised to 98 to 102° C. under stirring where the pressure inside the reactor was found to be 10 MPa (gauge pressure). Next, the reaction mixture was kept under stirring for 4 hrs at 98 to 102° C., and then cooled to 5 to 35° C. to give 60.82 g of a solution of N-acetylmethionine in 1-methyl-2-pyrrolidinone. The high-performance liquid chromatography analysis of the solution showed that the yield of N-acetylmethionine based on 3-(methylthio)propionaldehyde was 46.77%.

### Comparative Example 4

A reactor made of stainless steel equipped with a thermocouple, stirrer, gas-supplying line, and liquid-supplying line was charged with 5.26 g (0.050 mol) of 3-(methylthio)propionaldehyde, 3.01 g (0.050 mol) of acetamide, 1.04 g (0.0013 mol) of a complex of palladium bromide(II) and triphenylphosphine, 1.54 g (0.0175 mol) of lithium bromide, 0.05 g (0.00051 mol) of sulfuric acid and 51.50 g of 1-methyl-2-pyrrolidinone (100% by mass of the total amount of 1-methyl-2-pyrrolidinone), and the resulting mixture was stirred and the gas-phase of the reactor was pressurized with carbon monoxide gas by 10 MPa (gauge pressure). Then, the temperature of the reaction mixture was raised to 98 to 102° C. under stirring where the pressure inside the reactor was found to be 10 MPa (gauge pressure). Next, the reaction mixture was kept under stirring for 4 hrs at 98 to 102° C., and then cooled to 5 to 35° C. to give 61.42 g of a solution of N-acetylmethionine in 1-methyl-2-pyrrolidinone. The high-performance liquid chromatography analysis of the solution showed that the yield of N-acetylmethionine based on 3-(methylthio)propionaldehyde was 52.22%.

### Comparative Example 5

A reactor made of stainless steel equipped with a thermocouple, stirrer, gas-supplying line, and liquid-supplying line was charged with 3.01 g (0.050 mol) of acetamide, 0.33 g (0.0013 mol) of palladium bromide(II), 1.54 g (0.0175 mol) of lithium bromide, 0.05 g (0.00051 mol) of sulfuric acid and 51.50 g of 1-methyl-2-pyrrolidinone (100% by mass of the total amount of 1-methyl-2-pyrrolidinone), and the resulting mixture was stirred and the gas-phase of the reactor was pressurized with carbon monoxide gas by 10 MPa (gauge pressure). Then, the temperature of the reaction mixture was raised to 98 to 102° C. under stirring where the pressure inside the reactor was found to be 10 MPa (gauge pressure). Next, 5.26 g (0.050 mol) of 3-(methylthio)propionaldehyde was added to the reactor over 2 hrs at the reaction mixture was kept under stirring for 4 hrs at 98 to 102° C., and then cooled to 5 to 35° C. to give 60.42 g of a solution of N-acetylmethionine in 1-methyl-2-pyrrolidinone. The high-performance liquid chromatography analysis of the solution showed that the yield of N-acetylmethionine based on 3-(methylthio)propionaldehyde was 38.38%.

### Comparative Example 6

A reactor made of stainless steel equipped with a thermocouple, stirrer, gas-supplying line, and liquid-supplying line was charged with 5.26 g (0.050 mol) of 3-(methylthio)propionaldehyde, 3.01 g (0.050 mol) of acetamide, 0.33 g (0.0013 mol) of palladium bromide(II), 0.068 g (0.050 mol) of triphenylphosphine, 1.54 g (0.0175 mol) of lithium bromide, 0.05 g (0.00051 mol) of sulfuric acid and 51.50 g of 1-methyl-2-pyrrolidinone (100% by mass of the total amount of 1-methyl-2-pyrrolidinone), and the resulting mixture was stirred and the gas-phase of the reactor was pressurized with carbon monoxide gas by 6 MPa (gauge pressure). Then, the temperature of the reaction mixture was raised to 118 to 122° C. under stirring where the pressure inside the reactor was found to be 6 MPa (gauge pressure). Next, the reaction mixture was kept under stirring for 12 hrs at 118 to 122° C., and then cooled to 5 to 35° C. to give 59.70 g of a solution of N-acetylmethionine in 1-methyl-2-pyrrolidinone. The high-performance liquid chromatography analysis of the solution showed that the yield of N-acetylmethionine based on 3-(methylthio)propionaldehyde was 8.74%.

1. A method for producing N-acylamino acid of formula (I): 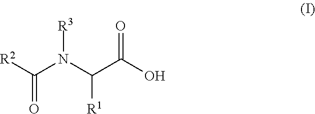
wherein R¹, R² and R³ are the same or different and each independently represents a hydrogen atom, a substituted or unsubstituted hydrocarbyl group, or a substituted or unsubstituted heterocyclic group,
which comprises supplying an aldehyde compound of formula (II): 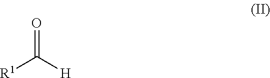
wherein R¹ is as defined above,
an amide compound of formula (III): 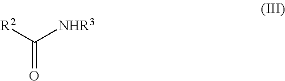
wherein R² and R³ are as defined above,
and a solvent to a reactor in which a solvent, a palladium compound, a halide compound, and carbon monoxide had been charged.

2. A method according to claim 1, wherein the amount of the solvent that had been charged in the reactor is 50 to 90% by mass of the total amount of the solvent to be supplied and the charged solvent.

3. A method according to claim 1 or 2, wherein the halide compound is a halide compound selected from the group consisting of an alkali metal halide, ammonium halide, and a quaternary ammonium halide.

4. A method according to claim 1 or 2, wherein the halide compound is an alkali metal halide.

5. A method according to claim 1, wherein the palladium compound is palladium halide.

6. A method according to claim 1, wherein the solvent is a aprotic polar solvent.

7. A method according to claim 1, wherein the solvent is 1-methyl-2-pyrrolidinone.

8. A method for producing N-acylamino acid of formula (I) as defined above, which comprises bringing a catalytic amount of a palladium compound and a halide compound into contact in a solvent under an atmosphere of carbon monoxide to produce a catalyst mixture, and supplying the aldehyde compound of formula (II) as defined above, and the amide compound of formula (III) as defined above to the resulting mixture.

9. A method according to claim 8, wherein R¹ is a hydrogen atom, an alkyl group, an alkylthioalkyl group, an alkenyl group, an aryl group, or an aralkyl group, R² and R³ are the same or different and independently represent a hydrogen atom, an alkyl group, an aryl group, or an aralkyl group.

10. A method according to claim 1, wherein R¹ is a hydrogen atom, a (C1-C6)alkyl group, a (C1-C4)alkyl-thio-(C1-C4)alkyl group, a (C2-C4)alkenyl group, a (C6-C12)aryl group, or a (C7-C14)aralkyl group, R² and R³ are the same or different and each independently represent a hydrogen atom, a (C1-C6)alkyl group, a (C6-C12)aryl group, or a (C7-C14)aralkyl group.

11. A method according to claim 1, wherein R¹ is a 2-methylthioethyl group, R² is a methyl group, and R³ is a hydrogen atom.
